(19) United States
(12) Patent Application Publication
(10) Pub. No.: US 2008/0249195 A1
(43) Pub. Date: Oct. 9, 2008

(54) POLYGLYCEROL/BENZOIC ACID-BASED EMULSIFIER

(75) Inventors: Anton A. Mentlik, Dallas, TX (US); Robert Foley, Dallas, TX (US); James Swanzy, Arlington, TX (US)

Correspondence Address: FULBRIGHT & JAWORSKI L.L.P., 600 CONGRESS AVE., SUITE 2400, AUSTIN, TX, 78701, US

(73) Assignee: Mary Kay Inc., Dallas, TX (US)

(21) Appl. No.: 12/046,007

(22) Filed: Mar. 11, 2008

Related U.S. Application Data
(60) Provisional application No. 60/894,364, filed on Mar. 12, 2007.

(51) Int. Cl. B01F 3/08; C07C 69/76

(52) U.S. Cl. 516/73; 560/112

(57) ABSTRACT

Disclosed is an emulsifier of formula (I):

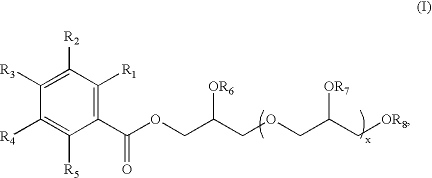

wherein: R₁ through R₅ are each independently H, —OH, alkyl, hydroxyalkyl, alkoxy and

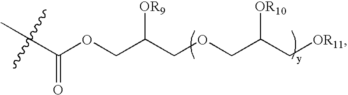

wherein R₉ through R₁₁ are each independently H or an alcohol protecting group; R₆ through R₈ are each independently H, an alkyl group, a hydroxyalkyl group, an alkoxy group, or an alcohol protecting group; and x and y are each independently an integer from 0 to 50; or a salt or hydrate thereof, provided that if R₁ through R₅ are all H, x is not 0.

### CROSS-REFERENCE TO RELATED APPLICATIONS

This application claims the benefit of U.S. Provisional Application Ser. No. 60/894,364, filed Mar. 12, 2007, the contents of which are incorporated by reference.

### BACKGROUND OF THE INVENTION

A. Field of the Invention

The present invention relates generally to compounds that can be used as emulsifiers.

B. Description of Related Art

A typical emulsion includes at least one hydrophilic phase and at least one hydrophobic phase that may be immiscible with one another. An emulsifier can be used to promote the formation of a stable homogenous mixture of a hydrophilic phase with a hydrophobic phase.

Esters of fatty acids with polyhydric alcohols (e.g., pentaerythritol, dipentaerythritol) and polyglycerol fatty acid esters have been used as emulsifiers in cosmetic and pharmaceutical formulations. Emulsifiers based on unsaturated or saturated fatty acids have been shown to produce emulsions that may have stability problems (see U.S. Pat. No. 5,902,590).

### SUMMARY OF THE INVENTION

The inventors address the problem associated with previous emulsifiers by providing an emulsifier that includes a core structure having a benzoic acid-based molecule linked to a polyglycerol-based molecule via an ester linkage. In one non-limiting aspect of the present invention there is disclosed an emulsifier that includes the following formula (I):

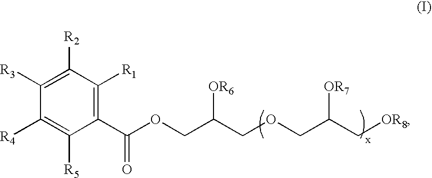

wherein:
R₁ through R₅ are each independently H, —OH, alkyl, hydroxyalkyl, alkoxy and

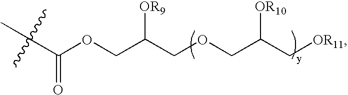

wherein R₉ through R₁₁ are each independently H or an alcohol protecting group; R₆ through R₈ are each independently H, an alkyl group, a hydroxyalkyl group, an alkoxy group, or an alcohol protecting group; and x and y are each independently an integer from 0 to 200; or a salt or hydrate thereof, provided that if R₁ through R₅ are all H, x is not 0. In certain aspects, x and y can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, or more or any range or integer therein. In particular embodiments, x and y can each independently be 1 to 20, 1 to 10, 1 to 5, or 3 to 5. x and y can be equal in certain aspects. In certain aspects, the emulsifier of the present invention does not include a fatty acid group. In other aspects, the emulsifier can include a fatty acid group. The emulsifier of the present invention can have a hydrophilic-lipophilic balance (HLB) value of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.8, 19.9, 20.0 or more or any integer or range derivable therein.

In certain aspects of the present invention, the emulsifier is comprised in an emulsion. The emulsion can include a hydrophilic phase and a hydrophobic phase. The emulsifier can be soluble within the hydrophilic or hydrophobic phase. The emulsifier can bind to a portion of the hydrophilic phase and a portion of the hydrophobic phase. The bond can be a hydrogen bond. In certain embodiments, the hydrophilic phase comprises water and the hydrophobic phase comprises an oil. Non-limiting examples of oils include silicone oils, mineral oils, oils of plant origin, synthetic oils, volatile oils, non-volatile oils, or ester oils. The emulsion can be a two-phase or three-phase emulsion. The two-phase emulsion can be a water-in-oil emulsion, an oil-in-water emulsion, a water-in-silicone emulsion, or a silicone-in-water emulsion. The three-phase emulsion can be a water-in-oil-in-water emulsion, an oil-in-water-in-oil emulsion, a water-in-silicone-in-water emulsion, or a silicone-in-water-in-silicone emulsion. In certain aspects, the emulsion can be a microemulsion. In certain non-limiting aspects, a water-in-oil emulsion can include 30 to 80% by weight of water and 2 to 50% by weight of oil (note that other percentages are contemplated, including those identified in other portions of the specification). In certain non-limiting aspects, an oil-in-water emulsion can include 5 to 30% by weight of oil and 40 to 85% by weight of water (note that other percentages are contemplated, including those identified in other portions of the specification). Hydrophilic phases of the emulsions can include water-soluble skin active ingredients that are known to those of ordinary skill in the art, including those identified throughout this specification. Similarly, the hydrophobic phases of the emulsions can include oil-soluble skin active ingredients that are known to those of ordinary skill in the art, including those identified throughout this specification. Emulsions of the present invention can include additional emulsifiers (e.g., at least two, three, for, five, six, seven, eight, etc. emulsifiers that are different from the emulsifier of the present invention), including mixtures thereof.

Another aspect of the present invention includes a composition comprising an emulsifier of the present invention. The compositions can be a cosmetic composition (e.g., topical skin care, hair care, fingernail care, etc.), a pharmaceutical composition (e.g. oral, topical, inhalant, injectable, etc.), a food composition, etc. The composition can include at least a second, third, fourth, fifth, sixth, seventh, or eight, or more emulsifiers that are different from the emulsifier of the present invention.

Also disclosed is a method of preparing a stable emulsion comprising: (a) obtaining a first and second liquid, wherein the first and second liquids are immiscible with one another; (b) obtaining an emulsifier of the present invention; and (c) mixing the first liquid, the second liquid, and the emulsifier of claim 1 to obtain a mixture, wherein the mixture is a stable emulsion. In certain embodiments, the first liquid is a hydrophilic liquid and the second liquid is a hydrophobic liquid. The first liquid can be added to the second liquid or the second liquid can be added to the first liquid. The hydrophilic liquid can be water. The hydrophobic liquid can be an oil. Non-limiting examples of oils include silicone oils, mineral oils, oils of plant origin, synthetic oils, volatile oils, or non-volatile oils. The method can further include obtaining at least a second, third, fourth, fifth, sixth, seventh, eight, or more, emulsifiers and mixing them with the first liquid, the second liquid, and the emulsifier of the present invention.

Also disclosed is a method of mixing two immiscible liquids comprising: (a) obtaining a first and second liquid, wherein the first and second liquids are immiscible with one another; (b) obtaining an emulsifier of the present invention; and (c) mixing the first liquid, the second liquid, and the emulsifier of the present invention, wherein a mixture of the first liquid, the second liquid, and the emulsifier is obtained. The resulting mixture can be homogenous mixture.

In yet another embodiment of the present invention there is disclosed a method of making an emulsion comprising reacting a compound of formula (II)

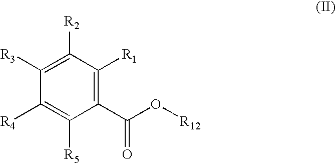

or a salt or hydrate thereof, with a compound of formula (III)

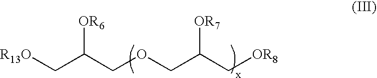

or a salt or hydrate thereof, wherein: R₁ through R₅ are each independently H, —OH, alkyl, hydroxyalkyl, alkoxy, or

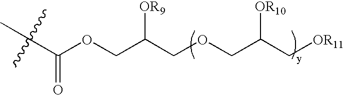

or a salt or hydrate thereof, R₉ through R₁₁ are each independently H or an alcohol protecting group and y is an integer from 0-200; R₁₂ is H or

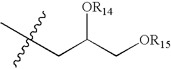

or a salt or hydrate thereof, R₁₄ and R₁₅ are each independently H or an alcohol protecting group, provided that at least one of R₁₄ and R₁₅ is H; R₆, R₇, R₈ and R₁₃ are each independently H or an alcohol protecting group, provided that at least one of R₆, R₇, R₈ and R₁₃ is H; and x is an integer from 0 to 200. The method can also include removing at least one alcohol protecting group.

It is contemplated that any embodiment discussed in this specification can be implemented with respect to any compound, method, or composition of the invention, and vice versa.

The term “alkyl” includes straight-chain alkyl groups, branched-chain alkyl groups, cycloalkyl (alicyclic) groups, alkyl heteroatom-substituted cycloalkyl groups, and cycloalkyl heteroatom-substituted alkyl groups.

The term “alkoxy” includes a group having the structure —OR, where R is an alkyl group. Non-limiting examples of alkoxy groups include —OCH₃, —OCH₂CH₃, —OCH₂CH₂CH₃, —OCH(CH₃)₂, —OCH(CH₂)₂, etc.

The term “hydroxyalkyl” includes an alkyl group having at least one hydroxy group.

The terms “about” or “approximately” are defined as being close to as understood by one of ordinary skill in the art, and in one non-limiting embodiment the terms are defined to be within 10%, within 5%, within 1%, or within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

The inventors have discovered that emulsifiers having a core structure of a benzoic acid-based molecule linked to a polyglycerol-based molecule via an ester linkage at the hydroxy (—OH group) of the benzoic acid molecule can produce stable emulsions. A benzoic acid molecule has the following structure:

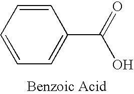

Benzoic acid can be produced by partial oxidation of toulene with oxygen. Benzoic acid is also commercially available from a variety of sources (e.g., Alfa Aesar, Ward Hill, Massachusettes USA).

A glycerol molecule has the following structure:

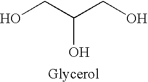

Glycerol can be synthesized from vegetable oil via treatment with methanol (CH₃OH) and a catalytic base such as potassium hydroxide (KOH). Glycerol is also commercially available from a variety of sources (e.g., MP Biomedicals, Solon, Ohio USA).

The following sections provide detailed non-limiting embodiments of the polyglycerol/benzoic acid-based emulsifiers and corresponding emulsions of the present invention

### A. Polyglycerol/Benzoic Acid-Based Emulsifier

In certain non-limiting aspects, an emulsifier of the present invention can include the following structure:

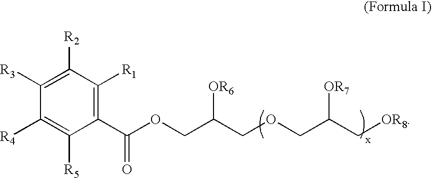

In non-limiting aspects, R₁, R₂, R₃, R₄, and R₅ can each independently be an H group, an —OH group, an alkyl group, a hydroxyalkyl group, an alkoxy group, or can have the following structure

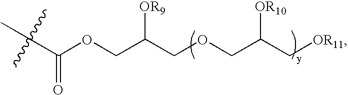

wherein R₉, R₁₀, and R₁₁, can each independently be an H group or an alcohol protecting group. R₆, R₇, and R₈ can each independently be an H group, an alkyl group, a hydroxyalkyl group, an alkoxy group, or an alcohol protecting group. x and y can independently be an integer of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 350, 400, 450, 500, 550, 600, 650, 700, 750, 800, 850, 900, 950, 1000, or more or any range or integer therein. The structures can also be a salt or hydrate of such structures as well. In certain embodiments, if R₁, R₂, R₃, R₄, and R₅ are all H, then x is not 0. In certain embodiments, x and y are independently an integer from 1 to 30, 1 to 20, 1 to 10, 1 to 5, or 3 to 5. In particular aspects, R₁ to R₅ are H.

The x and y integers and R groups can be varied to provide a molecule having a desired hydrophilic-lipophilic balance (“HLB”) (e.g., the balance between the hydrophilic and lipophilic portions of the molecule). Larger HLB value molecules can result in a molecule that can be more soluble in water and can be used as an oil-in-water emulsifier. Smaller HLB value molecules can result in a molecule that can be more soluble in oil and can be used as a water-in-oil emulsifier. By way of example only, the HLB value of a molecule of the present invention can be increased in certain aspects by increasing the x and/or y values, whereas the HLB value of the molecule can be decreased by decreasing the x and/or y values. In certain aspects, the HLB value of the molecules of the present invention can be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.8, 19.9, 20.0, or more, or any range or integer therein. Non-limiting examples of HLB value ranges of molecules of the present invention include 1 to 3, 3 to 6, 7 to 9, 8 to 18, 13 to 15, and 15 to 20.

The polyglycerol benzoate emulsifiers of the present invention can be made by using standard chemical synthesis methods that are known to those of ordinary skill in the art. For instance, one could add a benzoic acid molecule and a polyglycerin molecule having a desired chain length to a flask equipped with a Dean-Stark trap and condenser along with a common solvent and boiling chips. The mixture would be refluxed at the boiling point of the chosen solvent. The water generated by the reaction would condense and be captured in the trap. The end point could be calculated by the amount of water collected in the trap versus the theoretical yield.

Modifications to the molecules of the present invention are also contemplated. Non-limiting examples of such modifications include the addition or removal of lower alkyls such as methyl, ethyl, propyl, or substituted lower alkyls such as hydroxymethyl or aminomethyl groups; carboxyl groups and carbonyl groups; hydroxyls; nitro, amino, amide, and azo groups; sulfate, sulfonate, sulfono, sulfuydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; and substitution of a phenyl by a larger or smaller aromatic group. Alternatively, in a cyclic or bicyclic structure, hetero atoms such as N, S, or O can be substituted into the structure instead of a carbon atom. In certain aspects, the modifications can be used to change the hydrophilicity or hydrophobicity of the emulsifier (e.g., including additional hydrophilic or hydrophobic groups or tails to the ring portion of the emulsifier).

### B. Emulsions of the Present Invention

An emulsion typically includes a mixture of at least two immiscible substances. For example, one substance (the dispersed phase) can be dispersed in the other substance (the continuous phase). By way of example, one substance can be a hydrophilic substance (e.g., water) and the other a hydrophobic substance (e.g., oil). In a water-in-oil emulsion, the water phase is dispersed in the oil phase. In an oil-in-water emulsion, the oil phase is dispersed in the water phase.

Emulsions of the present invention can be prepared by a number of methods known to those skilled in the art (see, e.g., Sjoblom, 2005; Mollet et al., 2001). By way of example only, an emulsion can be prepared in a mixing tank and refined and stabilized by passage through a colloid mill or homogenizer. Another non-limiting method includes shaking together the two liquids or by adding one phase drop by drop to the other phase with some form of agitation, such as irradiation by ultrasonic waves of high intensity. Emulsification can also be accomplished by means of emulsifying machines or by other methods known to those of ordinary skill in the art (e.g., the Continental method, the English method, the Bottle Method, or the Beaker method).

A microemulsion is typically a smaller and more thermodynamically stable form of an emulsion. It is typically a dispersion of two immiscible liquids in which the individual droplets of the dispersed phase have an average radius less than about ¼ of the wavelength of light. Typically, in a microemulsion the dispersed phase droplets are less than about 1,400 A. Additionally, microemulsions tend to appear more transparent or translucent than regular emulsions. Microemulsions of the present invention can be prepared by a number of methods known to those skilled in the art (see, e.g., U.S. Pat. No. 4,146,499). For example, the microemulsions can be formed from the components discussed throughout this document with the aid of a stirrer or blending equipment. Other commercially available emulsifying equipment providing mechanical agitation can also be used to prepare the microemulsions.

1. Hydrophilic Phase

The hydrophilic phase of an emulsion of the present invention can be 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99%, or any integer or range therein, by weight or volume of the emulsion. The hydrophilic phase can include hydrophilic ingredients that are known to those of ordinary skill in the art (see, e.g., International Cosmetic Ingredient Dictionary, 10^{th} Ed., 2004) and those that are disclosed throughout the specification. Non-limiting examples of such ingredients include water, polyols, or mixtures thereof. In various embodiments, the polyol can be glycerin, propylene glycol, butylene glycol, pentylene glycol; polyethylene glycol, ethoxylated glycerin, lower weight alcohols, etc., or mixtures thereof.

2. Hydrophobic Phase

The hydrophobic phase of an emulsion of the present invention can be 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99%, or any integer or range therein, by weight or volume of the emulsion. The hydrophobic phase can include hydrophobic ingredients that are known to those of ordinary skill in the art (see, e.g., International Cosmetic Ingredient Dictionary, 10^{th} Ed., 2004) and those that are disclosed throughout the specification. Non-limiting examples include oils, fatty acids, fatty alcohols, waxes of natural or synthetic origin, etc.), hydrocarbon solvents, film formers, silicones, silicone polymers, fluorinated solvents, etc.

Non-limiting examples of oils that can be used in the context of the present invention include: oils of plant origin (e.g., sweet almond oil, coconut oil, castor oil, jojoba oil, olive oil, rapeseed oil, groundnut oil, sunflower oil, wheat germ oil, maize germ oil, soya oil, cotton oil, lucerne oil, poppy oil, marrow oil, evening primrose oil, millet oil, barley oil, rye oil, safflower oil, canelle nut tree oil, passionflower oil, hazelnut oil, palm oil, shea butter, apricot stone oil, Alexandria laurel tree oil, sysymbrium oil, avocado oil, calendula oil, etc.); modified plant oils (e.g., products known under INCI designations Apricot Kernel Oil PEG-6 esters, Olive Oil PEG-6 esters, etc.); oils of natural origin (e.g., perhydrosqualene, squalene, etc.); mineral oils (e.g., liquid paraffin, mineral oils originating from petroleum fractions such as isoparaffins having a boiling point between 300 and 400° C., etc.); synthetic oils (e.g., fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propylene glycol dicaprylate, ester derivatives of lanolic acid, such as isopropyl lanolate, isocetyl lanolate, monoglycerides, triglycerides such as glycerol triheptanoate, alkylbenzoates, isoparaffins, polyalphaolefins, polyolefins, such as polyisobutylene, synthetic isoalkanes such as isohexadecane, isododecane, perfluorinated oils, silicone oils, etc.). Non limiting examples of silicone oils include dimethyl polysiloxanes, methylphenylpolysiloxanes, amine-modified silicones, fatty acid-modified silicones, alcohol-modified silicones, alcohol- and fatty acid-modified silicones, polyether group-modified silicones, epoxy-modified silicones, fluoro group-modified silicones, cyclic silicones, alkyl group-modified silicones, etc.). Other non-limiting oils known to those of skill in the art that can also be used in the context of the present invention can be found in International Cosmetic Ingredient Dictionary, 10^{th} Ed., 2004.

3. Co-Emulsifiers

Emulsions of the present invention can also include a co-emulsifier. A co-emulsifier can include surfactants that can be used in combination with the emulsifiers disclosed in the present invention to form stable emulsions. Non-limiting examples of surfactants that can be used include nonionic, cationic, anionic, and zwitterionic surfactants (See McCutcheon's (1986); U.S. Pat. Nos. 5,011,681; 4,421,769; 3,755,560). For instance, examples of co-emulsifiers include glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof (See International Cosmetic Ingredient Dictionary, 10^{th} Ed., 2004).

4. Stabilizers

Emulsions of the present invention can also include a stabilization agent. Stabilization agents can be an ingredient that aids in stabilizing emulsions or compositions of the present invention. Non-limiting examples of stabilization agents include those known to a person of ordinary skill in the art (see, e.g., International Cosmetic Ingredient Dictionary, 10^{th} Ed., 2004) and those disclose throughout the specification. Examples include hydrogenated castor oil, plant waxes (e.g., beeswax and carnauba wax, etc.), stearic acid, magnesium stearate, aluminum stearate, hydrophobic silicas, polyethylene glycol-alkyl glycol copolymers, mineral waxes, etc.

### C. Additional Ingredients

Emulsions of the present invention can include additional ingredients. Non-limiting examples of additional ingredients include cosmetic ingredients and pharmaceutical active ingredients.

1. Cosmetic Ingredients

The CTFA International Cosmetic Ingredient Dictionary and Handbook (2004) describes a wide variety of non-limiting cosmetic ingredients that can be used in the context of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

a. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

b. Moisturizers

Non-limiting examples of moisturizing agents that can be used with the compositions of the present invention can be found in the International Cosmetic Ingredient Dictionary, 10^{th} Ed., 2004. Examples include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

c. Emollients

Non-limiting examples of emollients include, but are not limited to, vegetable oils, mineral oils, silicone oils, synthetic and natural waxes, medium chain triglycerides, petrolatum, lanolin, aluminum magnesium hydroxide stearate (which can also function as a water repellent), and fatty acid esters. Non-limiting examples of vegetable oils include safflower oil, corn oil, sunflower seed oil, and olive oil.

d. Antioxidants

Non-limiting examples of antioxidants include, but are not limited to, acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

e. Thickening Agents

Thickening agents, including thickener or gelling agents, include substances which that can increase the viscosity of a composition. Thickeners include those that can increase the viscosity of a composition without substantially modifying the efficacy of the ingredients within the composition. Thickeners can also increase the stability of the compositions of the present invention. Non-limiting examples of additional thickeners that are known to those of ordinary skill in the art can be used in the context of the present invention (e.g., U.S. Pat. Nos. 5,087,445; 4,509,949; 2,798,053; International Cosmetic Ingredient Dictionary, 10^{th} Ed., 2004). Examples include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. The silicon containing compound can be a silicone oil such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in International Cosmetic Ingredient Dictionary, 10th edition, 2004 as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

2. Pharmaceutical Active Agents

Pharmaceutical active agents are also contemplated as being useful with the emulsion compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### D. Amount of Ingredients

A person of ordinary skill would recognize that emulsion compositions of the present invention can include any number of combinations of the ingredients (e.g., emulsifier, hydrophilic ingredients, hydrophobic ingredients, co-emulsifiers, stabilizers, cosmetic ingredients, pharmaceutical ingredients, etc.) discussed throughout this specification. The concentrations of the ingredients can vary. In non-limiting embodiments, for example, the emulsion compositions may include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or more, or any range or integer derivable therein, of at least one of the ingredients mentioned throughout the specification and claims. In non-limiting aspects, the percentage can be calculated by weight or volume of the total emulsion composition. A person of ordinary skill in the art would understand that the concentrations can vary depending on the desired effect of the emulsion composition and/or on the product into which the emulsion composition is incorporated into.

### E. Equivalents

Known and unknown equivalents to the ingredients discussed throughout this specification can be used with the emulsion compositions and methods of the present invention. The equivalents can be used as substitutes for the ingredients. The equivalents can also be used to add to the methods and emulsion compositions of the present invention. A person of ordinary skill in the art would be able to recognize and identify acceptable known and unknown equivalents to the ingredients without undue experimentation.

### F. Products

The emulsion compositions of the present invention can be incorporated into products. Non-limiting examples of products include cosmetic products, food-based products, pharmaceutical products, etc. By way of example only, non-limiting cosmetic products include sunscreen products, sunless skin tanning products, hair products, fingernail products, moisturizing creams, skin benefit creams and lotions, softeners, day lotions, gels, ointments, foundations, night creams, lipsticks, cleansers, toners, masks, or other known cosmetic products or applications. Additionally, the cosmetic products can be formulated as leave-on or rinse-off products.

### G. Kits

Kits are also contemplated as being used in certain aspects of the present invention. For instance, emulsion compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the emulsion composition. In other embodiments, the container can be squeezed (e.g., metal, laminate, or plastic tube) to dispense a desired amount of the emulsion composition. The emulsion composition can be dispensed as a spray, an aerosol, a liquid, a fluid, or a semi-solid. The containers can have spray, pump, or squeeze mechanisms. A kit can also include instructions for employing the kit components as well the use of any other emulsion compositions included in the container. Instructions can include an explanation of how to apply, use, and maintain the emulsion compositions.

### EXAMPLES

The following examples are included to demonstrate certain non-limiting aspects of the invention. It should be appreciated by those of skill in the art that the techniques disclosed in the examples which follow representative techniques discovered by the inventor to function well in the practice of the invention. However, those of skill in the art should appreciate that changes can be made in the specific embodiments which are disclosed and still obtain a like or similar result without departing from the spirit and scope of the invention.

### Example 1

### Method of Preparing a Polyglycerol Benzoate Emulsifier

A person of ordinary skill in the art would be able to prepare a polyglycerol benzoate emulsifier by using common chemical synthesis procedures. For instance, one could add a benzoic acid molecule and a polyglycerin molecule having a desired chain length to a flask equipped with a Dean-Stark trap and condenser along with a common solvent and boiling chips. The mixture would be refluxed at the boiling point of the chosen solvent. The water generated by the reaction would condense and be captured in the trap. The end point could be calculated by the amount of water collected in the trap versus the theoretical yield.

### Example 2

A non-limiting example of a water-in-oil emulsion of the present invention is illustrated in Table 1.

TABLE 1*

| Phase | Ingredient** | % Concentration (by weight) |
|---|---|---|
| A | Mineral Oil | 10.0 |
| | Silicone Copolyol | 3.0 |
| | Polyglycerol-4 Benzoate | 3.0 |
| | Cosmetic Esters | 5.0 |
| B | Water | Q.S. |
| | Electrolyte (NaCl or MgSO₄) | 0.5-1.0% |
| | Humectant | 2.0 |
| | Preservatives | 1.0 |
| | TOTAL | 100 |

*This water-in-oil emulsion can be made by using the following process. Mix the hydrophobic phase ingredients in a main vessel until the mixture is uniform. Mix the hydophilic phase ingredients in a separate vessel until the mixture is uniform. Subsequently, add the hydrophilic phase mixture to the hydrophobic phase mixture slowly with a moderate propeller agitation. Homogenize the entire batch for five (5) minutes.
**Phases A and B are the hydrophobic and hydrophilic phases, respectively. Note that additional water soluble ingredients can be added and the amount of water can vary in this non-limiting water-in-oil emulsion.

A non-limiting example of an oil-in-water emulsion of the present invention is illustrated in Table 2.

TABLE 2*

| Phase | Ingredient** | % Concentration (by weight) |
|---|---|---|
| A | Mineral Oil | 10.0 |
| | Glyceryl Stearate | 3.0 |
| | Cetyl Alcohol | 1.0 |
| | Polyglycerol Benzoate | 3.0 |
| | Cosmetic Esters | 5.0 |
| B | Water | Q.S. |
| | Humectant | 2.0 |
| | Preservatives | 1.0 |
| | TOTAL | 100 |

*This oil-in-water emulsion can be made by using the following process. Mix the hydrophobic phase ingredients in a main vessel and heat to 70° C.-75° C. Mix the hydophilic phase ingredients in a separate vessel and heat to 70° C.-75° C. Subsequently, add the hydrophobic phase mixture to the hydrophilic phase mixture slowly with a moderate propeller agitation. Allow the mixture to cool to less than 30° C. while mixing.
**Phases A and B are the hydrophobic and hydrophilic phases, respectively. Note that additional water soluble ingredients can be added and the amount of water can vary in this non-limiting water-in-oil emulsion.

Note that the stability of the above emulsions can be determined by methods that are known to those of ordinary skill in the art (see, e.g., Sjoblom, 2005; Mollet et al., 2001). For instance, the stability of the water-in-oil emulsion can be determined by freezing the emulsion overnight and evaluating the emulsion as it thaws. A sample can also be subjected to elevated temperatures for several weeks to confirm stability. Stability of the oil-in-water emulsion can be determined by subjecting the emulsion to elevated temperatures for several weeks and evaluating the emulsion.

All of the emulsifiers, emulsions, compositions, and methods disclosed and claimed in this specification can be made and executed without undue experimentation in light of the present disclosure. While the emulsifiers, compounds, compositions, and methods of this invention have been described in terms of certain embodiments, it will be apparent to those of skill in the art that variations may be applied to the emulsions, compounds, compositions, and methods and in the steps or in the sequence of steps of the method described herein without departing from the concept, spirit and scope of the invention. More specifically, it will be apparent that certain emulsifiers and compounds which are both chemically and physiologically related may be substituted for the compounds described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 2,798,053
U.S. Pat. No. 3,755,560
U.S. Pat. No. 4,146,499
U.S. Pat. No. 4,421,769
U.S. Pat. No. 4,509,949
U.S. Pat. No. 5,011,681
U.S. Pat. No. 5,087,445
U.S. Pat. No. 5,902,590
CTFA International Cosmetic Ingredient Dictionary and Handbook, 2004.
International Cosmetic Ingredient Dictionary, 10^{th} Ed., 2004
McCutcheon, In: Detergents and Emulsifiers, North American Ed., 1986.
Mollet et al., In: Formulation Technology. Emulsions, Suspension, Solid Form, 2001.
Sjoblom, In: Emulsions and Emulsion Stability, 2^{nd} Ed., 2005.

1. An emulsifier of formula (I): 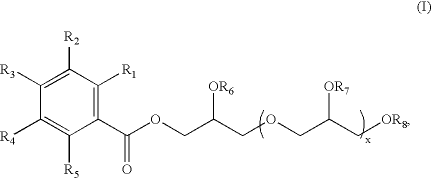
wherein:
R₁ through R₅ are each independently H, —OH, alkyl, hydroxyalkyl, alkoxy and 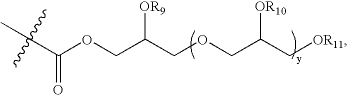
wherein R₉ through R₁₁ are each independently H or an alcohol protecting group;
R₆ through R₈ are each independently H, an alkyl group, a hydroxyalkyl group, an alkoxy group, or an alcohol protecting group; and
x and y are each independently an integer from 0 to 50;
or a salt or hydrate thereof,
provided that if R₁ through R₅ are all H, x is not 0.

2. The emulsifier of claim 1, wherein x and y are each independently 1 to 20.

3. The emulsifier of claim 1, wherein x and y are each independently 1 to 5.

4. The emulsifier of claim 1, wherein x and y are equal.

5. The emulsifier of claim 1, wherein the emulsifier has a hydrophilic-lipophilic balance (HLB) value of 1 to 3.

6. The emulsifier of claim 1, wherein the emulsifier has a hydrophilic-lipophilic balance (HLB) value of 3 to 6.

7. The emulsifier of claim 1, wherein the emulsifier has a hydrophilic-lipophilic balance (HLB) value of 7 to 9.

8. The emulsifier of claim 1, wherein the emulsifier has a hydrophilic-lipophilic balance (HLB) value of 8 to 18.

9. The emulsifier of claim 1, wherein the emulsifier has a hydrophilic-lipophilic balance (HLB) value of 13 to 15.

10. The emulsifier of claim 1, wherein the emulsifier has a hydrophilic-lipophilic balance (HLB) value of 15 to 20.

11. The emulsifier of claim 1, wherein the emulsifier does not include a fatty acid group.

12. The emulsifier of claim 1, wherein the emulsifier is comprised in an emulsion.

13-26. (canceled)

27. The emulsifier of claim 12, wherein the emulsion is a water-in-oil, water-in-oil-in-water, oil-in-water, oil-in-water-in-oil, or oil-in-water-in-silicone emulsion.

28. The emulsifier of claim 12, wherein the emulsion is a microemulsion.

29-32. (canceled)

33. A method of preparing a stable emulsion comprising:
(a) obtaining a first and second liquid, wherein the first and second liquids are immiscible with one another;
(b) obtaining the emulsifier of claim 1; and
(c) mixing the first liquid, the second liquid, and the emulsifier of claim 1 to obtain a mixture,
wherein the mixture is a stable emulsion.

34-40. (canceled)

41. The method of claim 33, further comprising obtaining a co-emulsifier and mixing the co-emulsifier with the first liquid, the second liquid, and the emulsifier of claim 1.

42-44. (canceled)

45. A method of making the emulsifier of claim 1, comprising reacting a compound of formula (II) 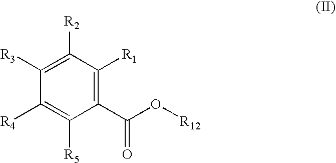
or a salt or hydrate thereof,
with a compound of formula (III) 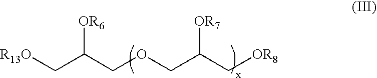
or a salt or hydrate thereof,
wherein:
R₁ through R₅ are each independently H, —OH, alkyl, hydroxyalkyl, alkoxy, or 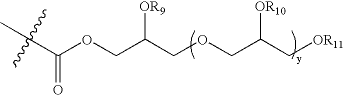
or a salt or hydrate thereof,
R₉ through R₁₁ are each independently H or an alcohol protecting group and y is an integer from 0-200;
R₁₂ is H or 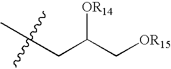
or a salt or hydrate thereof,
R₁₄ and R₁₅ are each independently H or an alcohol protecting group, provided that at least one of R₁₄ and R₁₅ is H;
R₆, R₇, R₈ and R₁₃ are each independently H or an alcohol protecting group, provided that at least one of R₆, R₇, R₈ and R₁₃ is H; and
x is an integer from 0 to 200.

46. The method of claim 45, wherein R₁ through R₅ are each H, R₁₂ is H, R₁₃ is H, R₆ through R₈ are each an alcohol protecting group, and x is 1.

47. The method of claim 45, further comprising removing at least one alcohol protecting group.

48-58. (canceled)
